(19) United States
(12) Patent Application Publication
(10) Pub. No.: US 2026/0188444 A1
(43) Pub. Date: Jul. 2, 2026

(54) GRAPHICAL USER INTERFACES AND SYSTEMS FOR PRESENTING AND ANALYZING LARGE PATIENT DATA SETS

(71) Applicant: THE JOHNS HOPKINS UNIVERSITY, Baltimore, MD (US)

(72) Inventors: Antony Rosen, Pikesville, MD (US); Christopher Mecoli, Baltimore, MD (US); Willie C. Stewart, JR., Columbia, MD (US); Hannah P. Cowley, Baltimore, MD (US); Matthew Chapman, Laurel, MD (US); Brant Chee, Laurel, MD (US)

(21) Appl. No.: 18/868,417

(22) PCT Filed: May 22, 2023
(86) PCT No.: PCT/US2023/023071

§ 371 Date: Nov. 22, 2024

Related U.S. Application Data
(60) Provisional application No. 63/344,958, filed on May 23, 2022.

(51) Int. Cl. G16H 10/60

(52) U.S. Cl. CPC G16H 10/60

(57) ABSTRACT

A method for presenting a graphical user interface (GUI) on a user device including a display and an input mechanism, includes accessing raw patient data on the user device, pre-processing the raw patient data to generate visualization data of a set of patient data records for each of multiple panel views, receiving a first input selecting a first panel view of the panel views, and displaying, in the GUI presented on the display, the set of patient data records in the first panel view. The method further includes receiving a second input selecting a subset of the patient data records, filtering the pre-processed set of patient data records, on the user device and based on the second input, to obtain a filtered subset of patient data records, and displaying, in the GUI, the filtered subset of patient data records based on the visualization data.

### CROSS-REFERENCE TO RELATED APPLICATIONS

This application claims the benefit of U.S. Patent Application No. 63/344,958, filed on May 23, 2022. The disclosure of the prior application is considered part of, and is incorporated by reference in, the disclosure of this application.

### TECHNICAL FIELD

This document generally describes technology related to providing improved graphical user interfaces (GUIs) on computing devices for visualization and analysis of patient data sets.

### BACKGROUND

GUIs are interfaces through which users are able to interact with computing devices and applications that are being executed thereon. For example, GUIs provide graphical presentations of information and graphical elements (e.g., icons, virtual buttons, visual indicators, textboxes, sliders, radio buttons, menus) that a user is able to interact with (e.g., select, activate) through one or more input devices (e.g., mouse, keys/buttons, scroll wheel, trackball, touch/presence-sensitive surface, microphone, camera). GUIs have been implemented across a variety of different computing devices, such as desktop computers, laptop computers, and mobile computing devices (e.g., smartphone, tablets, wearable computing devices).

GUIs have also been implemented to dynamically obtain and present information from remote computer systems (e.g., cloud computing systems), or to present and interact with information stored locally on a user device.

### SUMMARY

Analysis of patient data often requires visualization of large amounts of data, and manipulation of the data to understand various aspects or parameters. Each patient record can include many separate parameters or characteristics (i.e., is multi-dimensional), and investigating the relevance of each parameter with respect to a primary diagnosis or additional aspects can be unwieldy and difficult, both computationally and logistically. Because of the large size and multi-dimensionality of patient data sets, determining relevant correlations in the data can be tedious, and some patterns that require identification of particular data subgroups may not be identified.

Additionally, conventional tools for investigating patient data require an initial hypothesis be formulated for testing and provide “hypothesis driven” visualizations.

This document generally describes technology for presenting patient data content in a GUI with specialized features and process flows on a client computing device to enable efficient identification of patterns and trends in the data. The specialized features of the GUI enable display of multi-dimensional patient information for identification of relevant patterns that take into account multiple aspects of available patient records. The GUI displays the data in various specialized graphical representations that a user can toggle between to facilitate the identification of trends in the data and in data subgroups. The specialized GUI facilitates an enhanced user experience process flow that allows the user to navigate large amounts of data to identify inferences and connections in the data. The specialized GUI also includes options for filtering the data based on relevant clinical parameters, selecting patient population subgroups to investigate commonalities in patient populations, and visualizing large amounts of data in continually updated graphical displays for efficient exploration of data patterns to identify clinically relevant trends.

The process flows in the GUI enable the display of patient data in response to a pre-existing hypotheses (“hypothesis generated” visualizations), while also allowing serendipitous discovery by allowing the user to change between various connected visualizations. For example, a user can select a subgroup in one display view, which results in the automatic presentation of new patterns in other display views to which the user can navigate. The GUI enables the exploration of the data in ways that allow multiple dimensions of the patient records to be displayed to and understood by the user concurrently and intuitively.

For example, patient data is displayed in a specialized GUI on the client device with options to allow the user to view the data in different graphical representations such as a time series plot, onset time plot, or cluster plot. The data includes relevant clinical parameters and information, and the GUI includes specialized filtering options based on clinical and diagnostic parameters to enable a user to quickly move between views of the data and data subsets. The ability to filter and change the graphical view of the data allows the user to identify trends and patterns in the patient data which may have clinical, diagnostic, or treatment relevance, and can enhance the user experience process flow in working with large amounts of patient data by focusing on smaller subsets of a patient population. For example, a user toggling between various views and specialized filters of patient data may identify that patients in a particular age group are correlated with a higher incidence of a type of cancer. This may lead the user to consider additional examination or testing of patient's in the age group. The ability to filter and change the graphical view of the data in the GUI also allows the user to visualize changes in groups of patients over a period of time, even when data in particular patient records is sparse. A variety of different views, process flows, and specialized filters of patient data can be presented in such GUIs.

A method for presenting a graphical user interface includes accessing raw patient data on a user device, where the user device includes a display and an input mechanism. The method also includes pre-processing the raw patient data to generate visualization data of a set of patient data records for each of multiple panel views, receiving, at the input mechanism, a first user input selecting a first panel view of the multiple panel views, and displaying, in a GUI presented on the display of the user device, the set of patient data records in the first panel view. Additionally, the method includes receiving, at the input mechanism, a second user input selecting a subset of the patient data records, filtering the pre-processed set of patient data records, on the user device and based on the second user input, to obtain a filtered subset of patient data records, and displaying, in the GUI, at least the filtered subset of patient data records based on the visualization data.

Such a method can optionally include one or more of the following features. In some implementations, the raw patient data, visualization data, and set of patient data records, are stored on the user device. In some implementations, the method can include updating metadata associated with each record of the filtered subset of patient data records based on the filtering. In some implementations, the method includes receiving, at the input mechanism, a third user input that selects a second panel view of the multiple panel views, generating for display, based on the visualization data, the filtered subset of patient data records in the second panel view, and displaying, in the GUI, the filtered subset of patient data records in the second panel view.

In some implementations, the first user input is a selection of a number of graphical indicators representing data records in the set of patient records displayed in the first panel view. In some implementations, displaying at least the filtered subset of patient data records includes displaying only data records represented by the selected plurality of graphical indicators. In some implementations, the method can include displaying, in the GUI and based on a third user selection received at the input mechanism, additional information related to a selected data record of the filtered subset of patient data records.

In some implementations, the second user input selects a filtering characteristic. In some implementations, displaying at least the filtered subset of patient data records includes displaying the set of patient data records with the filtered subset of patient data records indicated by a visual identifier.

In some implementations, the first panel view is one of a cluster view, a time series view, and an onset time view. In some implementations, the method includes determining a relationship or fit to displayed patient data records in at least one of the plurality of panel views, and displaying the determined relationship or fit with the displayed patient data records.

In some implementations, the set of patient data records includes one or more data records of patients having a first diagnosis. In some implementations, filtering the set of patient data records further includes determining a subset of data records of the one or more data records of patients having a first diagnosis that have a first common characteristic. In some implementations, the first common characteristic is having a second diagnosis, different from the first diagnosis. In some implementations, the first common characteristic is one of a genetic marker, a type of diagnosis, age, race, disease state, or presence of antibody.

In some implementations, the set of patient data records includes data records of patients with myositis, and the first panel view includes a cluster plot view. In some implementations, the second panel view includes a time of onset view. In some implementations, the filtered subset of patient data records includes data records of patients with myositis having a diagnosis of cancer. In some implementations, the filtered subset of patient data records includes data records of patients with myositis having a genetic marker. In some implementations, the filtered subset of patient data records includes data records of patients with myositis having an antibody.

In some implementations, the set of patient data records includes data records of patients with chronic obstructive pulmonary disease (“COPD”), and the first panel view includes a Sankey plot view. In some implementations, the second panel view includes a cluster plot view and/or a SAX visualization. In some implementations, the patient data records are filtered by exacerbations, comorbidities, medications, or demographics. In some implementations, a filtered subset of patient data records includes data records of patients with COPD who have experienced or are experiencing homelessness. In some implementations, a filtered subset of patient data records includes data records of patients with COPD who have been hospitalized a number of times.

In another implementation, a method of analyzing patient data includes accessing a set of patient data records stored on a user device, the set of patient data records including data records for number of patients having a first diagnosis, each data record including an time offset factor to align each of the plurality of patients with a common time zero, and the user device including a display and an input mechanism. The method also includes receiving, at the input mechanism, a first input of a first filtering criteria, determining a subset of the set of patient data records that include the first filtering criteria, associating each record of the subset of the set of patient data records with the first filtering criteria, displaying, in a GUI presented in the display of the user device, the set of patient data records in a first panel view based on the common time zero, with the subset of the set of patient data records indicated by a visual indicator, receiving, at the input mechanism, a second input to display the set of patient data records in a second panel view, and displaying, in the GUI, the set of patient data records in the second panel view, with the subset of the set of patient data records indicated by the visual indicator based on the association of each record with the first filtering criteria.

Such a method can optionally include one or more of the following features. In some implementations, the method also includes receiving, at the input mechanism, a third input of a second filtering criteria, determining a second subset of the subset of the set of patient data records that include the second filtering criteria, and associating the second subset with the second filtering criteria. In some implementations, the method includes generating, after associating each record with the first filtering criteria, for display the set of patient data records. In some implementations, generating for display includes generating data for display in each of first and second panel views. In some implementations, the method also includes displaying, upon receiving a user selection of the first panel view or the second panel view, the data for display in the selected first or second panel view. In some implementations, the method also includes updating the generated data for display based on receiving a further input.

In some implementations, the first panel view is a time series and the second panel view is a cluster plot. In some implementations, the method includes generating a best fit line associated with the data for display for inclusion with the time series. In some implementations, the method also includes generating, for display in the GUI, additional views showing aspects of the subset of the set of patient data records linked with the first filtering criteria, the additional views generated for each of the first and second panel views. In some implementations, the aspects linked with the first filtering criteria are predetermined.

In some implementations, the method also includes generating for display a pop-up panel for each record of subset of the set of patient data records, the pop-up panel including aspects for the record linked with the first filtering criteria. In some implementations, the method also includes displaying the pop-up panel for a record of the subset of the set of patient data records in response to a selection of the record in the first panel view. In some implementations, generating for display the pop-up panel further includes extracting from each data record a set of predetermined aspects linked with the first filtering criteria.

In some implementations, the time offset factor is based on a date of the first diagnosis. In some implementations, the time offset factor is based on a date of first symptoms associated with the first diagnosis.

In another implementation, a method for handling patient data for display on a user device includes displaying a subset of stored patient data; receiving a user request to filter the subset of stored patient data; updating the subset of stored patient data based on the received user request; generating for display the updated subset of stored patient data in a first and a second presentation mode; displaying the updated subset of stored patient data in the first presentation mode based on the generation; receiving a user request to display the updated subset of stored patient data in the second presentation mode; and displaying the updated subset of stored patient data in the second presentation mode based on the generation.

Such a method can optionally include one or more of the following features. In some implementations, the method includes re-generating for display the updated subset of stored patient data in the first and second presentation modes in response to receiving a second user request to filter the updated subset of stored patient data. In some implementations, the method includes determining, based on the second user request, whether to display only a filtered set of the updated subset of stored patient data, or the updated subset of stored patient data with the filtered set of the updated subset of stored patient data identified by visual indicators. In some implementations, the subset of stored patient data is stored in the user device.

In another implementation, a method of analyzing patient data includes accessing patient data records from a memory, the patient data records including data records for a number of patients having a first diagnosis, each record of the patient data records including an array of text strings describing aspects of a medical history; receiving a first input to filter the patient data records based on a first criteria including a first text string; comparing the first text string to at least one text string of the array of text strings for each record of the patient data records; determining, based on the comparison, a subset of the patient data records including the first text string; associating the subset of patient data records including the first text string with a first indicator; generating for display the subset of the patient data records including the first text string; receiving a second input to filter the subset of patient data records based on a second criteria including a second text string; comparing the second text string to at least one text string of the array of text strings for each record of the subset of patient data records associated with the first text string; determining a second subset of patient data records including the second text string among the subset of patient data records associated with the first text string; associating the second subset of patient data records including the second text string with a second indicator; and generating for display the second subset of patient data records including the second string.

Certain implementations may provide one or more advantages. In a first example, a single specialized GUI can be used to present and change between different filtered views of patient data that would otherwise be individually created and presented or presented across multiple separate GUIs or applications. Such features can improve the GUI on client computing devices, such as client computing devices with smaller form factor displays, like screens on mobile and wearable computing devices. For instance, by presenting multiple different data views within a single GUI, users can more efficiently access information and features on their devices, and can more efficiently analyze the data.

In a second example, computing devices can operate more efficiently by not needing to execute multiple applications concurrently to navigate and visualize the data, which can reduce memory usage and processing requirements. The specialized pre-processing in the software and generation of data for display in multiple visualizations or graphical presentations enables the GUI to rapidly change between selected visualizations without requiring additional processing. Updating the multiple visualizations in response to user selections enables the visualizations to be connected or linked to enable a user to maintain context during exploration of the presented data.

In a third example, arrangements and sequences by which graphical elements are presented in the specialized GUIs can provide more intuitive and efficient graphical presentations on client computing devices and more natural process flows for user navigation of the data. The presentation of data in multiple connected graphical representations, including time-based and non-time-based series, enables a user to toggle between multiple views to recognize and identify patterns in patient populations and sub-populations. The user can access answers to questions or hypotheses about data trends and patterns quickly, and can intuitively navigate the data to identify associations of interest. Additional views presented in the specialized GUI showing patient characteristics related to a currently selected patient filtering characteristic can provide a guide or prompt to a user of additional aspects that can be explored and analyzed, resulting in further recognition of trends.

In a fourth example, manipulation of the data by adding specialized filters and changing the presentation of the data in the GUI can provide more intuitive representations of complex data on client computing devices. The presentation of clinical parameters as filtering or selection criteria in the specialized GUI allows the user to interact with each of the parameters in the patient data set to identify and visualization data patterns and trends. The presentation and filtering by multiple layers of factors can allow the discovery of patterns in patient data and sub-groups of patients in the patient data that might not be easily discovered using traditional methods of analysis. The user can select various filtering criteria and view the patient data including or excluding the filtering criteria with a click of a button, instead of having to extract the filtering criteria from each patient data record and plot the data in a separate application. The ease with which the data is manipulated allows the user to review the data with relation to various parameters to identify patterns that might otherwise be obscured by the amount of patient data and number of parameters for each patient record.

In a fifth example, a single GUI can be provided through which a user can readily navigate between different types of content and content streams without having to lose their context. The intuitive presentation allows toggling between patient data so that a user to more easily identify correlations or commonalities in relevant patient subgroups without being distracted or confused by the large amounts of data represented. For instance, a user can view data in a first plot type or graphical representation, add a filter and view the filtered data in the first plot type, select a number of patient records in the filtered data and view in a second plot type. The user can then un-filter the data, or return to the first plot type by interacting with the GUI.

### DESCRIPTION OF DRAWINGS

FIG. 1 is a block diagram of an example system providing data to the client device for presentation in the GUI.

FIG. 2 is a flow chart of an example process of processing patient data for presentation in a GUI.

FIG. 3 is a flow chart of an example process of filtering patient data for presentation in a GUI.

FIG. 4 is a flow chart of an example process of determining data for display in a GUI.

FIGS. 5A-5E depict an example process of displaying and filtering patient data presented in the GUI.

FIGS. 6A-6D depict an example process of interacting with patient data presented in the GUI.

FIGS. 7A-7D depict another example process of display and filtering patient data presented in the GUI.

FIG. 8 is a flow chart of an example process of processing data at the client device for display in a GUI.

FIG. 9 is a flow chart of an example process of providing data from a server to a client device.

FIG. 10 is a flow chart of an example process of a client device for obtaining patient data from a server and filtering the patient data for display on a GUI.

FIG. 11 is a block diagram of example computing devices that are optionally available for use with particular embodiments of the GUI and the platform described herein.

Like reference symbols in various drawings indicate like elements.

### DETAILED DESCRIPTION

This document generally describes systems and methods for presenting patient data content in a GUI with specialized features and process flows on a client computing device to enable efficient identification of patterns and trends in the data. The GUI displays the data in various specialized graphical representations that a user can toggle between to facilitate the identification of trends in the data and in data subgroups. The specialized GUI facilitates an enhanced user experience process flow that allows the user to navigate large amounts of data to identify inferences and connections in the data. The specialized GUI also includes options for filtering the data based on relevant clinical parameters, selecting patient population subgroups to investigate commonalities in patient populations, and visualizing large amounts of data in continually updated graphical displays for efficient exploration of data patterns to identify clinically relevant trends.

The systems and methods for presenting patient data content in a GUI with specialized features and process flows provide multiple advantages. For example, the specialized features and process flows can improve the GUI on client computing devices, such as client computing devices with smaller form factor displays, like screens on mobile and wearable computing devices. The computing devices can operate more efficiently by not needing to execute multiple applications concurrently to navigate and visualize the data, which can reduce memory usage and processing requirements. The enhanced process flows of the specialized GUI can provide more intuitive and efficient graphical presentations on client computing devices and more natural process flows for user navigation of the data, and manipulation of the data by adding specialized filters and changing the presentation of the data in the GUI can provide more intuitive representations of complex data on client computing devices. Further, the specialized GUI described herein includes enhanced user experience process flows through which a user can readily navigate between different types of content and content streams without having to lose their context.

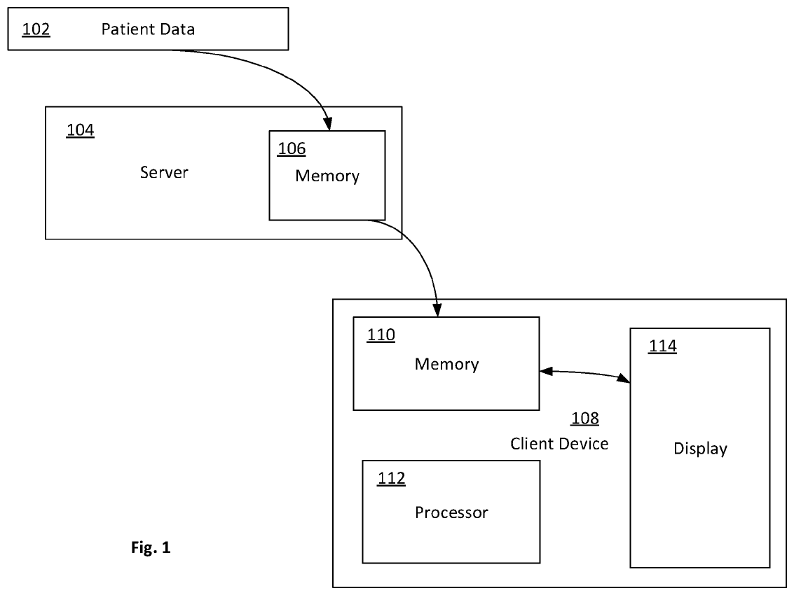

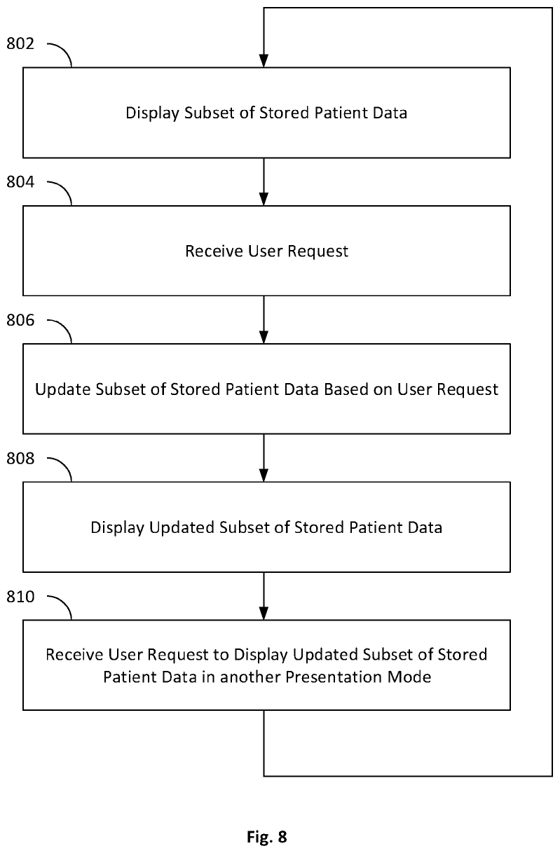

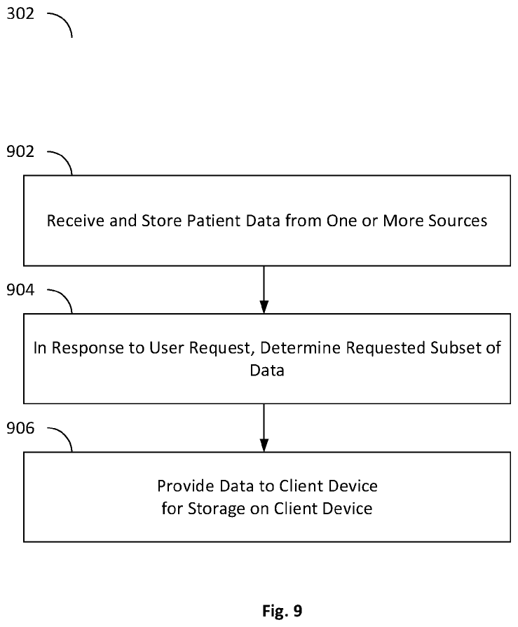

Referring to FIG. 1, an example process for providing patient data for analysis on a client device is illustrated. Patient data 102 is stored in a memory 106 of a server 104. A client device 108 includes a memory 110, a processor 112, and a display 114. The client device 108 requests patient data 102 from the memory 106 of the server 104. The patient data 102 is transmitted to the client device 108 and stored in the memory 110. Using analysis software stored in the processor 112, the client device 108 can present the patient data 102 in a GUI on the display 114. The process by which patient data 102 is transferred to the server 104 and between the client device 108 is described in FIGS. 8 and 9.

FIG. 9 is a flow chart of an example process of providing data from a server to a client device, for example, as illustrated in FIG. 1. At step 902, the server 104 receives patient data 102 from one or more sources, and stores the patient data 102 in a memory 106 of the server. At step 904, in response to a user request from a client device 108, the server 104 determines a requested subset of patient data 102. At step 906, the server 104 provides the subset of patient data 102 to the client device 108 for storage in a memory 110 of the client device.

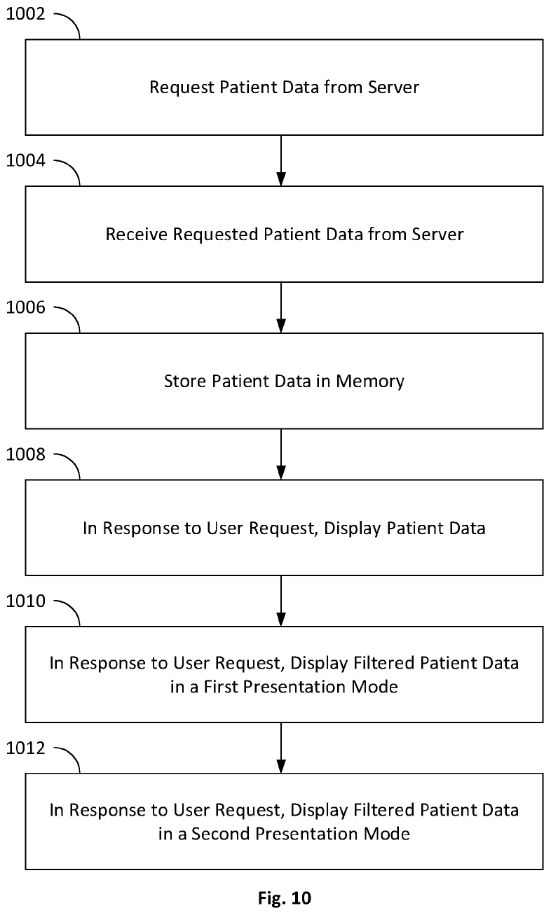

Referring to FIG. 10, an example process of a client device 108 for obtaining patient data 102 from a server 104 and filtering the patient data 102 for display on a GUI is illustrated. The method of FIG. 10 describes the method followed at the client device 108 for display of a subset of patient data 102 in the GUI for analysis by a user. At step 1002, the processor 112 of a client device 108 requests patient data 102 from the server 104. At step 100004, the client device 108 receives the requested patient data 102 from the server 104, and at step 1006, the client device 108 stores the patient data 102 in the memory 110. At step 1008, in response to a user request, the processor 112 generates the patient data 102 for display, and presents the patient data 102 in a GUI on the display 114 of the client device 108. At step 1010, in response to a user request, the client device 108 processor 112 displays filtered patient data 102 in a first presentation mode, and at step 1012, in response to a user request, the processor 112 displays the filtered patient data 102 in a second presentation mode.

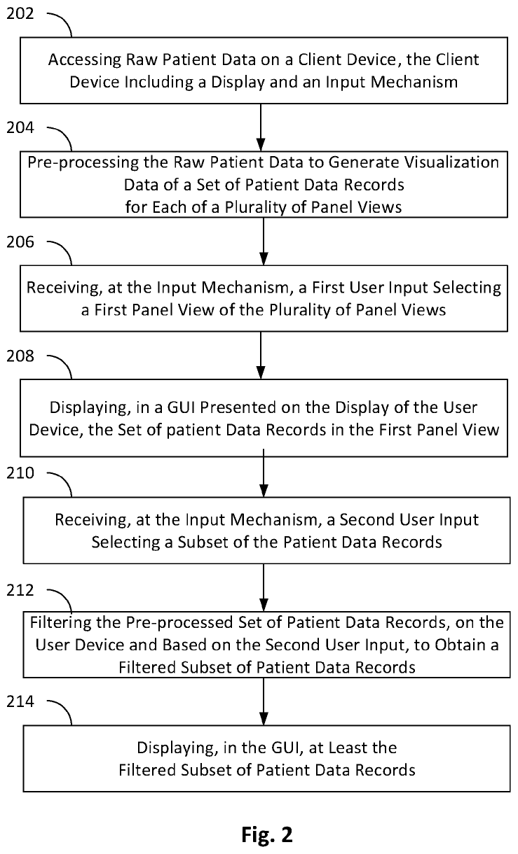

The presentation of the patient data 102 in the GUI in multiple panel views or presentation modes and the capability of providing a number of filtered views of the patient data 102 enables a user of the GUI at the client device 108 to easily explore the patient data 102 to identify trends and patterns that would otherwise require multiple separate data sets be used to prepare graphical representations for review and revision by a user. The GUI enables the user to quickly assess the data and revise the data selection to reveal associations between data sets and patient characteristics that can have clinical relevance. The mechanisms by which the GUI allows this exploration of the data are further described with reference to FIGS. 2-4, 5A-E, 6A-D, and 7.

Referring to FIG. 2, a flow chart of an example process of processing patient data for presentation in a GUI is illustrated. At step 202, raw patient data (for example, patient data 102) is accessed on a user device (for example user device 108). The user device includes a display and an input mechanism. The input mechanism can be a mouse, a keyboard, a touch pad, a microphone, a haptic feedback system, or any other suitable mechanism. At step 204, the raw patient data is pre-processed to generate visualization data of a set of patient data records for each of a number of multiple panel views. The panel views are graphical representations of data, for example, a time series, a logarithmic plot, a cluster plot, a bar graph, a pie chart, a geographic or map plot, or any other suitable plot. The pre-processing can occur in the processor of the user device, and the visualization data can be stored in the memory of the user device.

In some implementations, the raw patient data, visualization data, and set of patient data records, are stored on the user device. By downloading the patient data and processing the patient data on the user device, several advantages are realized. First, the time required for processing the data in the GUI in response to filtering or other user inputs can be decreased if the data is stored locally and does not need to be continuously accessed from the server. Second, security of the data can be improved by decreasing the number of times that the data is accessed via internet or wi-fi connections. Local storage of the patient records can further protect against data corruption or changes at the server level, ensuring continuity of the data for presentation in the GUI. In some implementations, the method can include updating metadata associated with each record of the filtered subset of patient data records based on the filtering. The metadata updated can be an entry in a data array stored locally. In some implementations, the updated data can mark the data as filtered data to be called in a further analysis through a user input in the GUI.

At step 206, a first user input is received at the input mechanism. The first user input selects, in a GUI, a first panel view of the multiple panel views. At step 208, the set of patient data records is displayed in the first panel view in the GUI presented on the display of the user device. In some implementations, the first user input is a selection of a number of graphical indicators representing data records in the set of patient records displayed in the first panel view. The selection of a number of graphical indicators can be by choosing to turn on one or more filters represented in the GUI, or by selecting the graphical indicators displayed in the panel view, for example by right or left clicking a mouse and drawing a box around a set of graphical indicators displayed in the panel view of the GUI. The filters can include filters based on patient diagnosis, patient bibliographic details, and patient outcomes, as well as additional medical information or medical history information. In some implementations, displaying at least the filtered subset of patient data records includes displaying only data records represented by the selected plurality of graphical indicators. In some implementations, the first panel view is one of a cluster view, a time series view, an onset time view, a Sankey plot view, and a SAX visualization view. In some implementations, the method includes determining a relationship or fit to displayed patient data records in at least one of the plurality of panel views, and displaying the determined relationship or fit with the displayed patient data records.

At step 210, a second user input is received at the input mechanism. The second user input selects a subset of the patient data records. At step 212, the pre-processed set of patient data records are filtered on the user device and based on the second user input, to obtain a filtered subset of patient data records. At step 214, the filtered subset of patient data records are displayed in the GUI on the display based on the visualization data. Additional data can also be displayed with the filtered subset of patient data. In some implementations, the filtered subset of patent data records are distinguished from the additional data by use of markers of different colors or shapes. In some implementations, the second user input selects a filtering characteristic. For example, filtering characteristics can include demographic information about patients, or more particular characteristics about one or more diseases and disorders. The filtering characteristics can include patient age at onset of a disorder, gender, race, type of primary diagnosis, diagnosis of cancer or a comorbidity, presence of one or more genetic markers or antibodies, or any other medical data. In some implementations, displaying at least the filtered subset of patient data records includes displaying the set of patient data records with the filtered subset of patient data records indicated by a visual identifier.

In some implementations, the method includes receiving, at the input mechanism, a third user input that selects a second panel view of the multiple panel views, generating for display, based on the visualization data, the filtered subset of patient data records in the second panel view, and displaying, in the GUI, the filtered subset of patient data records in the second panel view.

In some implementations, the method can include displaying, in the GUI and based on a third user selection received at the input mechanism, additional information related to one or more selected data records of the filtered subset of patient data records. For example, the third user selection can be a selection to view a subset of patient records as graphical indicators having a particular shape or color to differentiate them from other records. In one example, the third user selection can be a selection to display patient data records within the subset of patient records having a common characteristic, such as a common age at onset of cancer as a purple circle. Being able to select various parameters and patient characteristics and quickly visualize them within a larger patient population in the GUI can enable more efficient analysis of commonalities and trends among patients. In some implementations, the third user selection is a click on a single graphical indicator in the panel view, in response to which additional information associated with the patient record indicated by the graphical indicator can be viewed in a popup window. The additional information can be patient demographic information or patient diagnosis information. In some implementations, the additional information displayed depends on the panel view currently selected in the GUI.

In some implementations, the set of patient data records includes one or more data records of patients having a first diagnosis. In some implementations, filtering the set of patient data records further includes determining a subset of data records of the one or more data records of patients having a first diagnosis that have a first common characteristic. In some implementations, the first common characteristic is having a second diagnosis, different from the first diagnosis. In some implementations, the first common characteristic is one of a genetic marker, a type of diagnosis, age, race, disease state, or presence of antibody.

In some implementations, the set of patient data records includes data records of patients with myositis, and the first panel view includes a cluster plot view. In some implementations, the second panel view includes a time of onset view. In some implementations, the filtered subset of patient data records includes data records of patients with myositis having a diagnosis of cancer. In some implementations, the filtered subset of patient data records includes data records of patients with myositis having a genetic marker. In some implementations, the filtered subset of patient data records includes data records of patients with myositis having an antibody. In some implementations, instead of filtering a group of patient records, two separate groups of patient records can be compared and commonalities identified using the GUI. For example, a first group of patient data records associated with patients diagnosed with myositis can be compared to a second group of patient data records associated with patients diagnosed with a particular cancer, and the data sets can be visualized and explored in the GUI to identify commonalities in the data sets and differences.

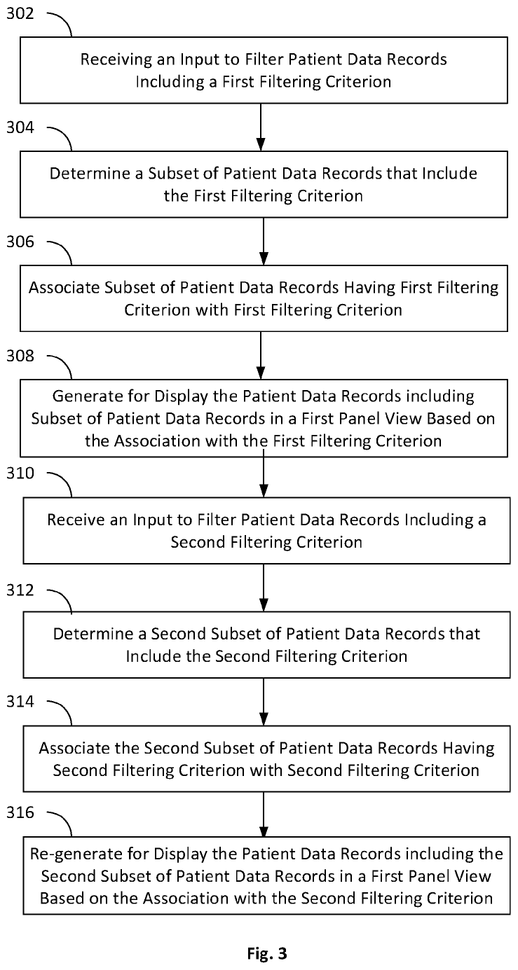

Referring to FIG. 3, a flow chart of an example process of filtering patient data for presentation in a GUI is illustrated. At step 302, a processor receives an input to filter patient data records including a first filtering criterion. At step 304, a subset of patient data records that each include the first filtering criteria is determined by the processor. At step 306, the subset of patient data records having the first filtering criteria are associated with the first filtering criteria. For example, an entry in a data array for each of the subset of patient data records can be updated. At step 308, the processor generates the patient data records including the subset of patient data records are generated for display in the GUI. The patient data records generated for display are determined based on the association of the patient data records with the first filtering criteria.

At step 310, the processor receives an input to filter the patient data records according to a second filtering criteria. At step 312, the processor determines a second subset of patient data records that include the second filtering criteria. At step 314, each record of the second subset of patient data records having the second filtering criteria is associated with the second filtering criteria. For example, an entry in a data array for each of the patient data records can be updated to indicate the association with the second filtering criteria. Finally, at step 316, the processor re-generates for display the patient data records including the second subset of patient data records in the first panel view based on association with the second filtering criteria.

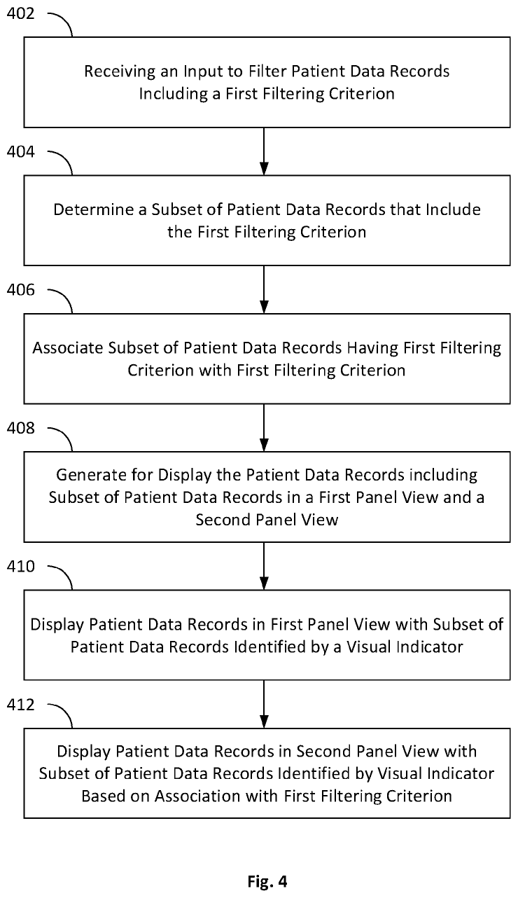

Referring to FIG. 4, a flow chart of another example process of determining data for display in a GUI is illustrated. The process for determining data for display can be executed by the processor of the client device in response to user inputs. At step 402, an input to filter patient data records including a first filtering criteria is received. At step 404, a subset of patient data records that include the first filtering criteria are determined. At step 406, the subset of patient data records that include the first filtering criteria are associated with the first filtering criteria or otherwise marked in the memory as being currently filtered data.

At step 408, the patient data records are generated for display, the records generated for display including the subset of patient data records having the first filtering criteria. The patient data records are generated for display in at least a first panel view and a second panel view, where each panel view is a different graphical display of the patient data records. The data records generated for display are continuously updated, linked, and re-processed or re-generated as the user interacts with the specialized GUI so that the user can seamlessly move between multiple presentations of the data without waiting for new displays of the data to be processed. For example, the panel views can be a time series, onset time view, a cluster plot, a logarithmic plot, a cluster plot, a bar graph, a pie chart, a geographic or map plot, a SAX visualization, a Sankey plot, or any other suitable graphical display of the data. The connected display visualizations enable serendipitous discovery by allowing the user to change between the various connected visualizations, without having to reproduce filters and selections for each display. For example, a user can select a subgroup in one display view, which results in the automatic presentation of new patterns in other display views to which the user can navigate. This can improve the user experience of exploring the data and can result in discovery of patterns in the patient data that might not have been easily observed using traditional hypothesis-generated visualization techniques.

At step 410, the patient data records are displayed in the first panel view with the subset of patient data records identified and differentiated from other patient data by a visual indicator. In some implementations, the visual indicator is a particular color marker or a shape marker that is different from the markers identifying other data. In some implementations, the user can select the visual indicator of the subset of patient data records. The use of colors and shapes to differentiate patient populations in a view can enable efficient pattern recognition by a user. The mechanisms and characteristics of the display of patient data can further be customizable based on the user or researcher's expertise, data set, and research interest. The questions being asked or investigated by the user can inform the navigation through the data, but the GUI can also be customized to enable intuitive visualization of patient records and characteristics that may be relevant to the particular questions. The GUI can be customized by incorporating additional patient data characteristics, views, or process flows related to the questions the user would like to investigate.

At step 412, the patient data records are displayed in the second panel view with the subset of patient data records identified by a visual indicator based on the association of the subset of patient data records with the first filtering criteria.

From the perspective of a user of the GUI, efficient analysis of the data and determination of trends and patterns in the data is improved because the GUI facilitates switching between views of the data and selection of various parameters for highlighting or filtering the data which may be clinically relevant. The movement between various views of the data appears seamless to the user because the data is stored locally and continuously generated for display in the various views on the backend. Accordingly, the user is able to explore aspects of the data quickly in order to identify novel patterns in the data.

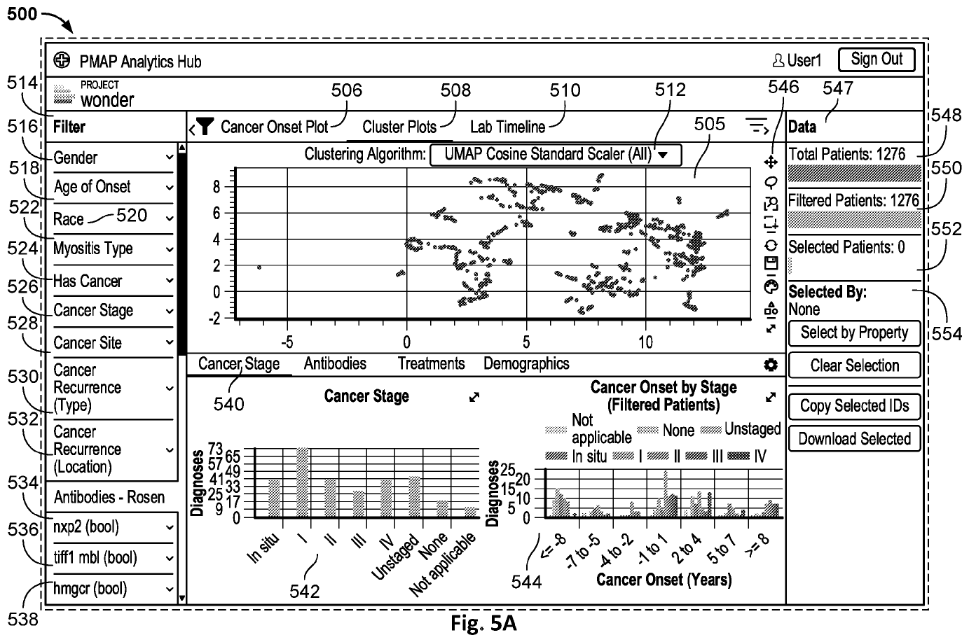

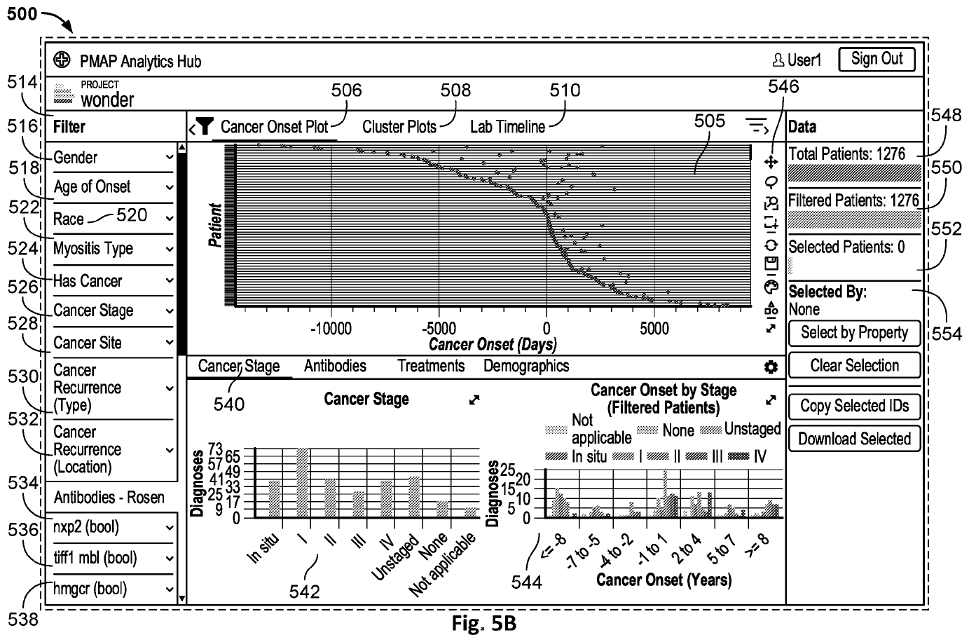

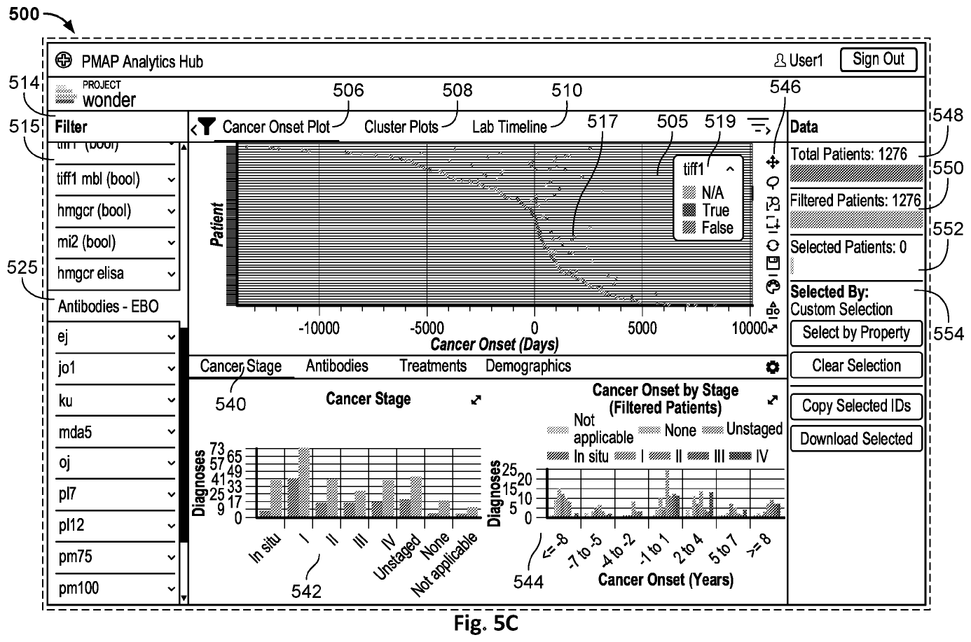

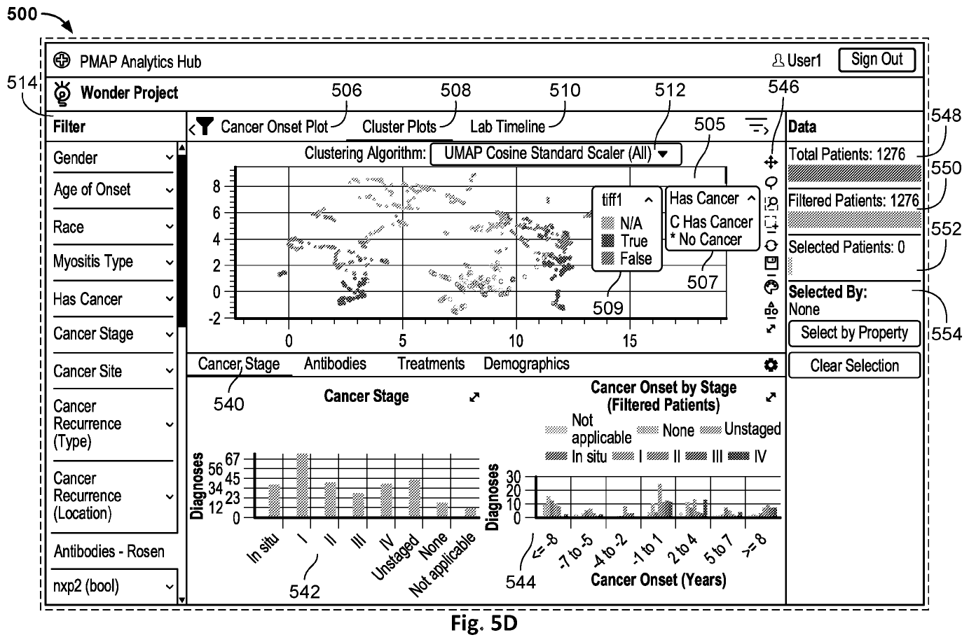

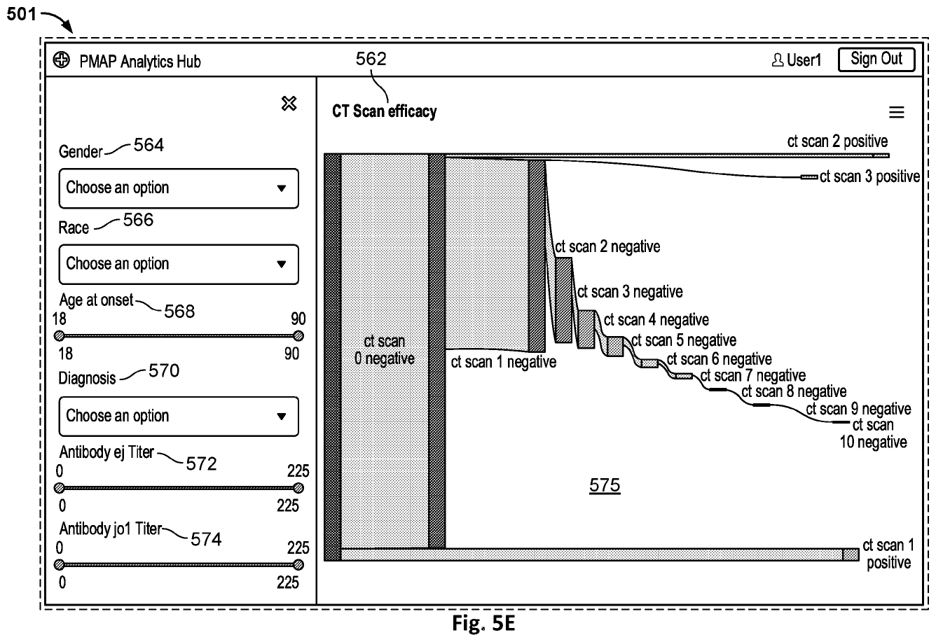

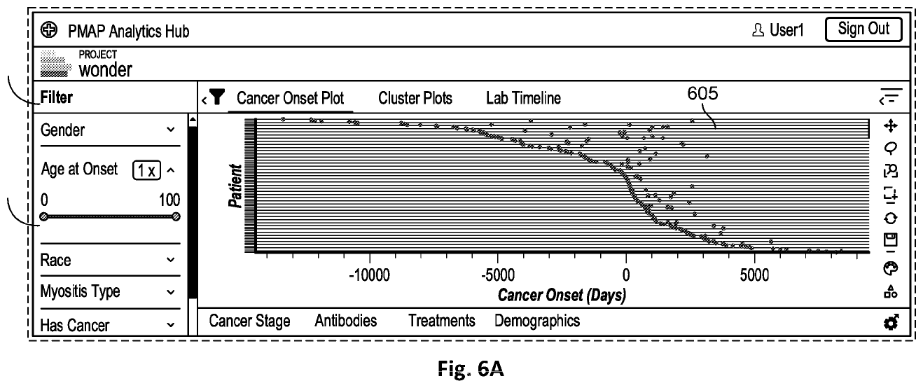

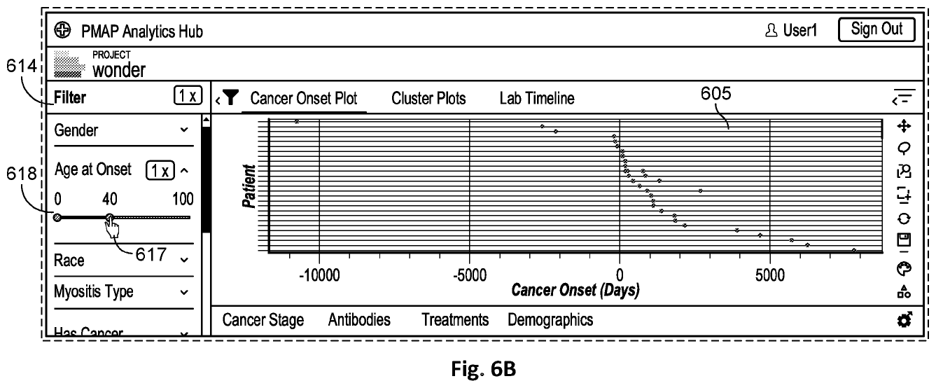

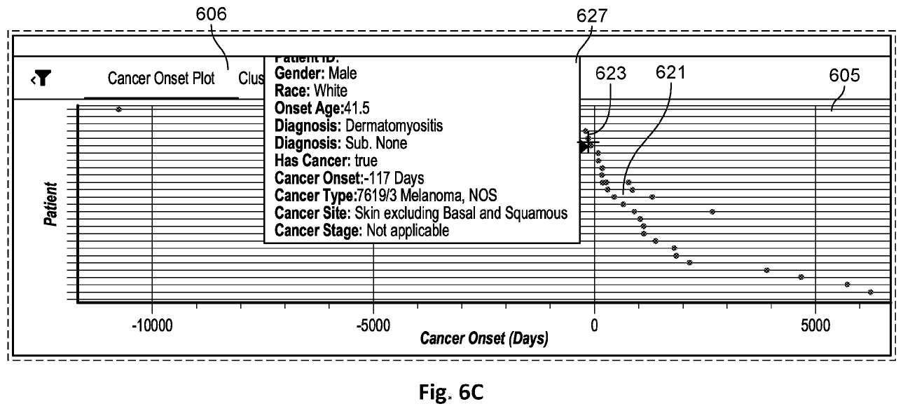

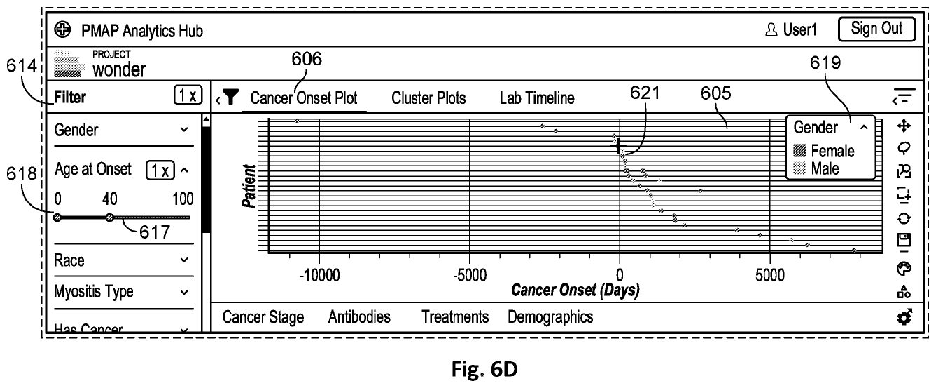

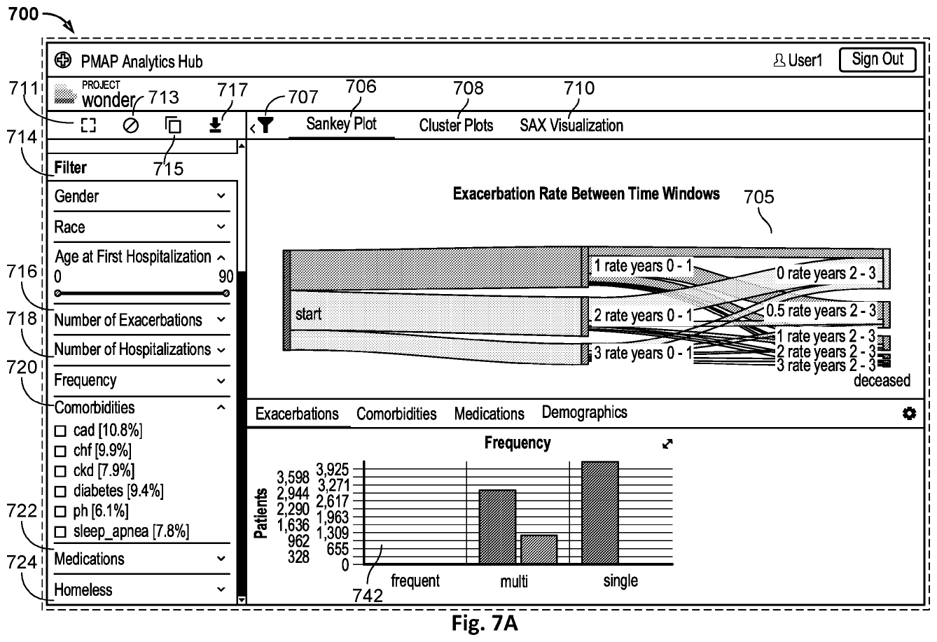

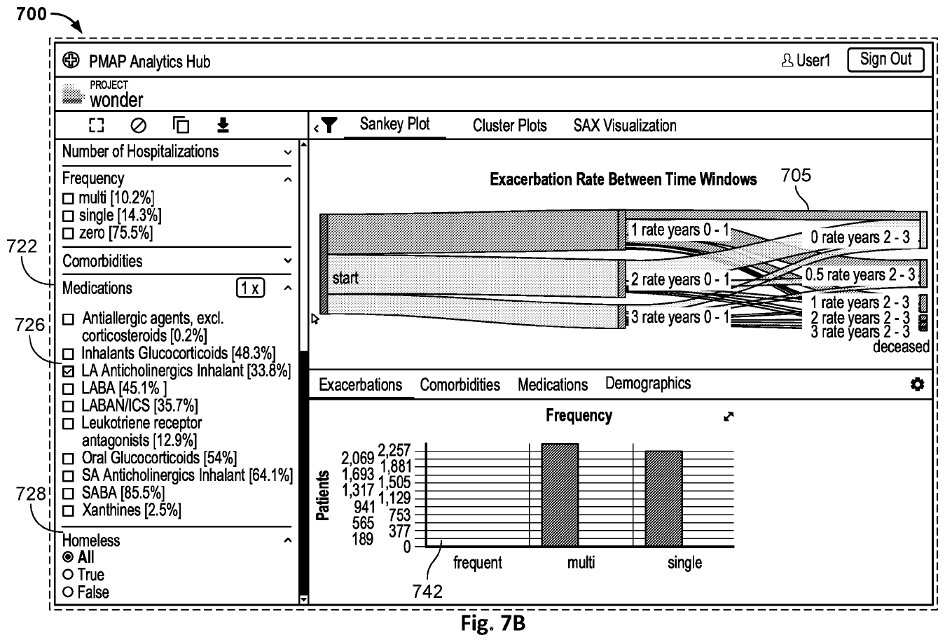

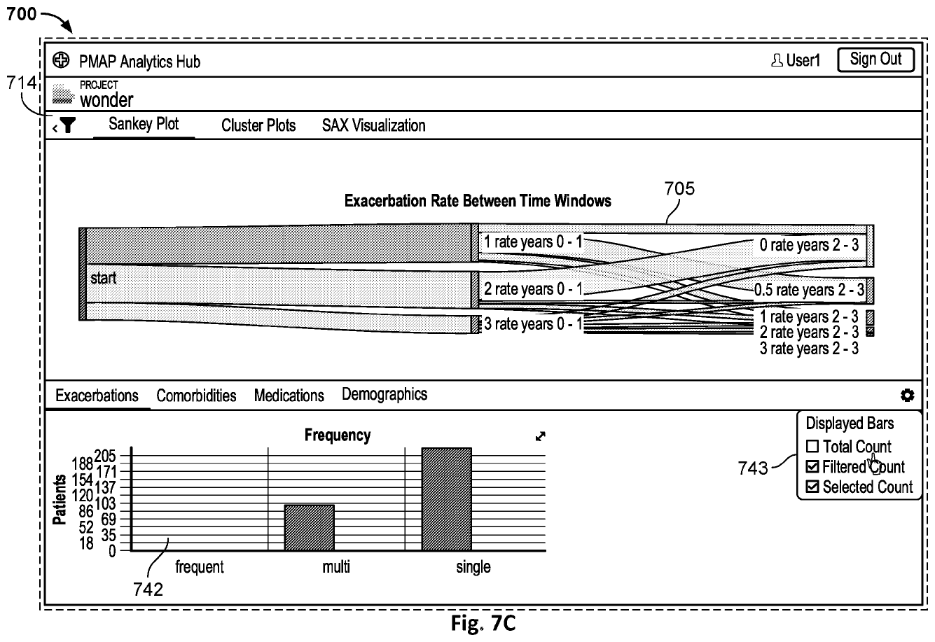

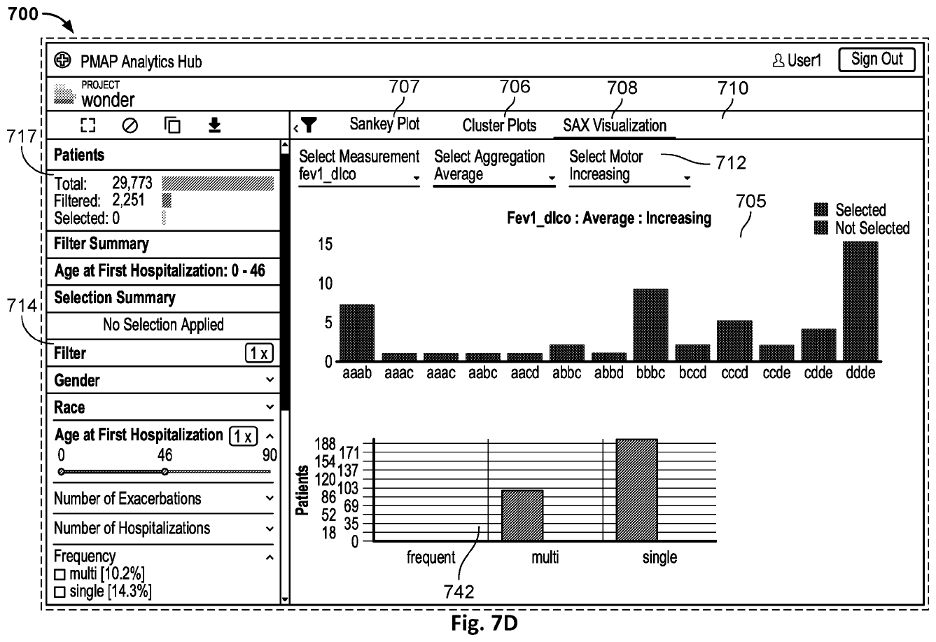

The GUI can be used to display and filter large amounts of patient data for patient populations characterized by multiple disease states. For example, FIGS. 5A-5E and 6A-6D illustrate example interactions with a GUI depicting patient data associated with patients diagnosed with myositis, and FIGS. 7A-D illustrate example interactions with a GUI depicting patient data associated with patients diagnosed with COPD.

FIGS. 5A-5E and 6A-D show examples of interactions with a GUI to display and filter patient data presented in the GUI for analysis of patterns and trends in patient data records. Referring to FIGS. 5A-5E, an example process of displaying and filtering patient data presented in the GUI is illustrated. The GUI 500 includes a first panel view 505 which displays the currently selected data graphically. The panel view 505 is also referred to as a presentation view or presentation mode in this document. The GUI 500 includes various patient parameter filters 514 that are selectable by a user to filter the displayed data in the GUI 500. The GUI further includes additional patient aspect views 542 and 544, and a navigation dashboard 547 to enable the user to make additional selections of the patient data or to clear the current selections and filters.

In FIG. 5A, the currently selected panel view 505 is a cluster plot 508. The GUI 500 includes additional selectable elements 506, 508, 510 that change the panel view 505 to another graphical view of the data. The other available panel views that can be selected are a cancer onset plot 506 or lab timeline 510. Other panel views may also be available, for example, a time series, logarithmic graph, geographical map, bar graph, pie graph, or any other suitable graphical representation of the data. The currently selected panel view 505 may have additional fits or display views that a user can select, for example through drop down menu 512. The user can select a clustering algorithm, a best fit algorithm, or an adjustment to the axes of the panel view 505.

Displaying the data in a cluster plot with various clustering algorithms enables a user to quickly interpret the displayed information to identify patient populations that respond to treatments or have similar characteristics. For example, a cluster plot with a linear regression clustering algorithm enables the identification of non-responders to a treatment, slow and rapid responders to a treatment, and outliers to the treatment responses of the patient population.

The GUI 500 filters 514 include options for the user to filter the data based on various patient parameters, such as gender 516, age of onset 518, race 520, disease type 522 (for example, myositis type), whether the patient has a cancer diagnosis 524, the stage of cancer 526 (for example, at a particular time after diagnosis with a primary diagnosis of myositis, at the time of diagnosis, or at another clinically relevant time point), the cancer site 528, whether there has been recurrence of the cancer after treatment, including the type of cancer recurring 530 and the location of recurrence 532, and the presence of various antibodies 534, 536, 538. Additional filtering criteria can also be presented to the user, for example, presence of genetic markers, family history of disease or illness, geographic location, or any other patient data. A user can select one or more of the filtering criteria to select a portion of the patient data to view in the graphical representation panel view 505.

Additional views of patient data aspects 542 and 544 are presented in the GUI 500 with the main panel view 505. The additional views 542, 544 can display aspects of the patient data that are related to the information selected and displayed in the panel view 505. The additional views 542, 544 can be pre-set or can be selected by the user from a list of options to view. Other aspects or graphical depictions of the data shown in additional views 542, 544 can be viewed by user selection of one of the options 540. For example, in FIG. 5A, the additional views show the cancer stage 542 and the cancer onset by stage 544 of the selected patient population. Other options 540 for view by the user include information related to antibodies in the selected patient population, treatments of the patients in the population, and patient population demographics.

The navigation dashboard 547 provides a view of what information is currently displayed in the panel view 505. The navigation dashboard 547 includes a representation of the total number of patients 548 in the patient data set, the filtered number of patients in the current view 550, the number of selected patients in the current view 552, and an indication of current filters applied to the data 554. In some implementations, the navigation dashboard 547 can also include quick toggle buttons for common or recently used data filters, an option for clearing all filters, removing the most recent filter, or undoing a most recent filter or change, and options for downloading, exporting, or saving the patient data and/or current views of one or more graphical representations in the GUI. A panel view dashboard 546 includes options for adjusting or altering the currently displayed panel view, for example, by changing the axes, magnifying portions of the panel view, saving the view, or changing the color or shape of the graphical indicators in the panel view.

The available options for selection and filtering of data, and options for moving between views of the data enables the user to explore the various parameters and aspects of the data by quickly adding and removing filters and selecting or unselecting groups of patients. The navigation dashboard 547 allows the user to recall the current context in which the data is being viewed so as not to become confused. In some implementations, the navigation dashboard 547 includes a navigation map of recently viewed filters or selections to provide “bread crumbs” to the user so that the user can remember what aspects have been previously explored or viewed and can return to these views as desired.

FIG. 5A illustrates the GUI 500 with no filters applied to the data. The patient data has been presented for display in the GUI 500 in a cluster plot panel view 505 without any filters applied. The patient records presented in the panel view 505 can be retrieved from the memory of the client device. In some implementations, the patient records are retrieved from a server external to the client device. Each of the patient records typically include numerous aspects associated with the patient's medical history and records of diagnosis and treatment. In FIG. 5A, the patient data records represented in the panel view 505 are records of patients having a diagnosis of myositis. The patient data records include various additional parameters related to the diagnosis, biographical details, and clinical history of each patient. The dates associated with each patient's diagnosis with myositis, and the dates associated with treatments or other diagnoses are offset in time so that all the patient records are aligned with a common time zero. In some implementations, the time offset factor is based on a date of the first diagnosis, for example, the diagnosis of each patient with myositis. In some implementations, the time offset factor is based on a date of first symptoms associated with the first diagnosis or the first hospitalization of the patient for a condition.

The time offset factor can be determined and used based on any date of interest to the research team, including the date of birth, date of death, date or dates of hospitalizations, or other relevant dates. In some implementations, more than one time offset factor is determined for each patient for which a record exists, and the user can select a desired time offset factor for use in display of the patient data. In some implementations, the user can toggle between different determined time offset factors in a display of patient data, for example by toggling between a first view using a time offset associated with the date of diagnosis and a second view using a time offset associated with a date of first hospitalization.

FIG. 5B shows the GUI 500 following the selection of the cancer onset plot 506 for display of the patient data in the panel view 505. A user can move between the GUI 500 of FIG. 5A displaying the data in a cluster plot in the panel view 505 to the FIG. 5B GUI 500 which presents the data in a cancer onset plot based on the time of the cancer diagnosis of patients. The user can observe based on the presentation of the data in the cancer onset plot that there is appears to be a population of patients diagnosed with myositis that are also diagnosed with cancer shortly after the myositis diagnosis. To further explore the correlation between patients diagnosed with myositis that are also diagnosed with cancer, the user can select additional filters of the data.

For example, in FIG. 5C, the data presented in the GUI 500 is further filtered by a first filtering criterion. In particular, FIG. 5C shows the GUI after filtering the patient data by the presence of the tiff1 antibody. The filter is selected from the antibodies filter 515 on the filtering panel 514. In response to the selection of the antibodies filter 515, the data in the panel display 505 is updated so that the graphical indicator for each patient indicates the presence or absence of the tiff1 antibody, or lack of data about the tiff1 antibody in the patient records. The graphical indicators are identified in the key 519.

In moving between the presentation of data shown in FIG. 5B to FIG. 5C, after receiving the filtering criteria selection input at the client device, the processor of the client device determines a subset of the set of patient data records that include the first filtering criteria, and associates each record of the subset of the set of patient data records with the first filtering criteria. The processor then generates for display the set of patient data records in a first panel view based on the common time zero, and presents the data in the GUI. In some implementations, the method includes generating the data for display in each of the available panel views. Generating the data for display after each selection by the user allows the user to quickly move between the various views of the data available on the GUI without having to wait for the generation of the data for presentation.

The user can further select a portion 517 of the patient data displayed in the panel view 505 for further analysis. In some implementations, the user can select the data by right or left clicking a mouse and dragging a box around a portion of the graphical indicators displayed in the panel view, selecting an input mechanism in the GUI and drawing a shape enclosing one or more of the graphical indicators, individually selecting graphical indicators, or any other suitable selection method. The selection of a portion 517 of the graphical indicators representing the patient data can cause the GUI to update the additional patient aspect views 542 and 544 to include representations of aspects of the selected portion 517 of the graphical indicators representing patient data in comparison to the total patient population. This can additionally aid the user in identifying trends or correlations in the subset of the patient population represented by the selected portion 517.

Additionally, when the user selects a portion 517 of graphical indicators, the navigation panel 548 can update to display the number of patients that have been selected in comparison to the total patient population. The display of the number of selected patients relative to the full number of patient records and the filtered number of patient records can aid the user in determining how statistically relevant the observations of the selected portion 517 is likely to be, and what clinical relevance the observations may have. Additionally, the navigation panel 548 can help the user to remember what filters and selections they made in the GUI 500 to arrive at the set of patient records that they are viewing in the panel view 505.

The user can select another panel view to toggle from the cancer onset plot 506 of FIG. 5C to the cluster plot 508 of FIG. 5D. A user input in the GUI 500 or to the client device requests that the set of patient data records be displayed in the second panel view, for example, the cluster plot view. The set of patient data records is presented to the user in the GUI with the subset of the set of patient data records indicated by the visual indicator based on the association of each record with the first filtering criteria.

As shown in FIG. 5D, the filters applied to the data are carried over to the newly selected panel view 505. The graphical indicators keep their coloring or shapes to differentiate between the patient records that have the filtered criteria of the tiff1 antibody and the patient records in which the tiff1 antibody is absent or the data is not available. The selected data from FIG. 5C is no longer selected in FIG. 5D. In some implementations, the GUI 500 provides the user with the option to preserve the selection of a portion of the data when the user selects a new panel view from the cancer onset plot 506, cluster plot 508 and lab timeline 510 or other available views.

The user can further select additional filtering criteria to apply to the panel view 505. In FIG. 5D, the panel view 505 displays a cluster plot 508 of the patient data which uses graphical indicators of different colors to indicate the presence or absence of the tiff1 antibody 509, and different shapes to indicate whether a patient represented by the graphical indicator has been diagnosed with cancer or has not 507. The ability of the GUI 500 to present data to the user which includes multiple layers of filtering through the use of distinct graphical indicators allows the user to explore and identify complex correlations between patient parameters, characteristics, diagnoses, and other aspects.

The GUI 500 can also include options for a user to further explore the additional patient aspect views 542 and 544. In FIG. 5E, an example of an additional screen 501 of the GUI of FIGS. 5A-D is illustrated. The additional screen 501 can be a pop-out displayed over the GUI 500 screen shown in FIGS. 5A-D. The additional screen 501, can show in greater detail and with additional filter options, one of the additional patient aspect views of the GUI 500. The additional screen 501 includes a graphical display 575 showing a representation of the data, a title 562 reminding the user which aspect they are viewing, and filter options. For example, the filter options can include gender, race, age at onset, diagnosis, and various antibody titers. In some implementations, additional filter options are available, such as geographic region, familial history, or any other relevant characteristic.

The additional screen 501 can be customizable based on the research interests of the user of the GUI. For example, in FIG. 5E, screen 501 illustrates the number of CT imaging scans performed to screen for cancer. Based on the displayed data, the user can observe that for the patient population being displayed, a large number of negative CT imaging scans are performed relative to the number of positive CT scans. The user may determine, based on this observation, that the treatment of the patient population includes extraneous cancer screening testing, which may be to the detriment of the patient and/or the healthcare system.

The GUI 500 provides numerous filtering options and viewing options for a user to explore large patient data sets. Users can interact with the GUI to limit or select smaller subsets of the patient data to identify trends and patterns. The ability of the user to quickly move between views of data or filtered data enables the user to explore patient characteristics without requiring the user to expend time and effort in preparing the data. This quick movement between views allows the user to “play” with the data and to investigate aspects of the data that current clinical evidence may not support as relevant. A user of the GUI is thus able to identify additional trends and patterns that would otherwise escape notice and observations that may be clinically relevant to diagnosis or treatment of certain patient populations.

The numerous filtering options and viewing options provide a multitude of possible interactions with a patient data set. FIGS. 5A-E illustrate one example of a user interaction with a patient data set. Another example process of interacting with patient data presented in the GUI is illustrated in FIGS. 6A-D. FIG. 6A shows a portion of the GUI of FIGS. 5A-E, with the panel view 605 displaying the cancer onset plot for a population of patients. The filter panel 614 includes multiple filter options, including a slider for selecting an age of onset of cancer 618. FIG. 6B shows a user moving the slider with a mouse indicator 617 to adjust the age of onset of cancer 618 in the filter panel 614. As the user adjusts the slider to change the filtered age of onset of cancer 618, the panel view 605 is adjusted to show only the graphical indicators representing patients that have the filter criteria. In this case, the filter criteria is an age of cancer onset of 40 or younger.

In some implementations, the processor of the client device can generate pop-ups, or other visual indicators for display for each patient record represented by a graphical indicator in the panel view. The pop-up display can provide more granular patient data to the user. The pop-up can include some or all data for each individual record. For example, in some implementations, the pop-up includes characteristics of a patient record that are linked with the currently selected filtering criteria. The pop-up can be displayed in the GUI for a patient record selected by the user in the first panel view. For example, a record can be selected by clicking on the graphical indicator representing the record in the panel view or by floating a cursor or mouse over the single dot representing a patient record in a plot. The pop-up can include demographic data, specific cancer type, cancer outcome, or any other data. In some implementations, the data displayed in the pop-up can be customized by the user, or it can be pre-determined based on the data displayed in the GUI. FIG. 6C shows an example of a pop-up 627 displayed in the GUI in response to the user clicking with a mouse indicator 623 on a single graphical indicator 621 displayed in the panel view 605. FIG. 6D illustrates the filtering of the patient data displayed in the panel view of FIGS. 6B and 6C, which is already filtered by the age of onset, by an additional filter of gender. The patient data 621 presented in the panel view 605 includes graphical indicators having two different colors to indicate the gender of the patient represented by each graphical indicator. As illustrated in FIG. 6D, multiple filters can be applied by a user and presented in the GUI to allow the user to investigate complex aspects of a disease or disorder and identify trends and patterns in subsets of the patient data records.

FIGS. 5A-5E and 6A-6D show example interactions with a GUI displaying patient data for patients with myositis. The GUI can be used with a variety of patient conditions and disease states, and the filtering options can be adjusted or changed to represent relevant data and characteristics of the particular patient population. FIGS. 7A-7D show additional example interactions with a GUI displaying patient data for patients with COPD. FIGS. 7A-7D illustrate an example process of displaying and filtering patient data presented in the GUI. FIGS. 7A-7D illustrate a GUI 700 displaying patient data associated with patients having COPD. The GUI 700 includes a first panel view 705 (also called the presentation view or presentation mode) which displays the currently selected data graphically. The GUI 700 includes various patient parameter filters 714 that are selectable by a user to filter the displayed data in the GUI 700. The GUI 700 further includes additional patient aspect views 742.

FIG. 7A illustrates a first view of the GUI 700. The first panel view 705 is a Sankey plot view. Viewing modes of the selected patient data are selected through user interaction with a Sankey plot element 706, a cluster plot element 708, and a SAX visualization mode element 710. The additional patient aspect view 742 shows a bar chart of occurrence of various exacerbations in the selected patient data. Other patient aspect views can be selected for display in the additional patient aspect view 742, including comorbidities, medications, and demographics. Additional functions are available using the buttons at the top left of the GUI. The additional functionalities include a sort by property button 711, clear filter selection button 713, copy selected patient identifiers button 715, and download selected de-identified patient identifiers button 717. A filter selection panel can be hidden or revealed using the filter button 707, and various patient parameter filters 714 are selectable by the user to filter and refine the displayed patient data. In addition to patient biographical data including gender, race, and age at first hospitalization with COPD, the data can be filtered by additional filtering aspects including number of exacerbations 716, number of hospitalizations 718, frequency of hospitalizations, specific comorbidities with COPD 720, specific medications taken for treatment of COPD 722, and homelessness status 724. Additional filters can be added and used in the GUI 700 based on available information in the patient data set and the particular disease condition being studied.

FIG. 7B shows the selection of a subset of patient data using the various patient parameter filters 714. In the medications filtering condition 722, the medication LA anticholinergics inhalant is selected 726 and patients of all homelessness statuses 728. The subset of patient data with these filtering conditions is displayed in the Sankey plot in the first panel view 705 and in the bar chart showing number of exacerbations in the additional patient aspect views 742. As shown, the medications filtering condition 722 lists medications that are represented in the patient data along with a percentage of the total number of patient data records reflecting that medication. The display of the percentage of patients that have a particular filtering quality aids the user in identifying relevant filtering conditions to explore in the GUI, and also aids the user in identifying irrelevant conditions or characteristics that lack sufficient data to be meaningful. In some implementations, each of the filtering options additionally show a number of patients with the particular filtering condition instead of or in addition to a percentage of patients. In some implementations, the filtering options show a percentage of patients out of the currently selected subset of patients that have the particular filtering condition. In some implementations, the filtering options show a percentage of patients out of the total number of available patient records that have the particular filtering condition.

FIG. 7C shows the selection of additional display options 743 in the additional patient aspect views 742. The additional display options 743 enable the user to make further selections to limit or filter the data displayed in the additional patient aspect views 742 to allow the user to better visualize the data or to manipulate the data to identify patterns and trends. FIG. 7C also illustrates that the panel showing the various patient parameter filters 714 can be condensed or closed to allow the GUI 700 to display the first panel view 705 and additional patient aspect views 742 in a larger format.

FIG. 7D shows the GUI 700 with the user selection of a SAX visualization mode element 710 from the options of Sankey plot element 706, cluster plot element 708, and SAX visualization mode element 710 in the first panel view 705. The SAX plot (displayed in first panel view 705 with user selection of SAX visualization mode element 710) can be further filtered or adjusted with the selection of additional management, aggregation, and motif options 712. In addition to the panel showing the various patient parameter filters 714, information related to the currently selected patient data records 717 is shown to aid the user in understanding the relevance of various filtering conditions to the entire available patient population. Showing the user the number of records currently displayed in the filtered view relative to the total available patient records can help the user to understand how large the filtered patient group being viewed is, and how representative the filtered patient group may be of the total patient population.

Referring to FIG. 8, a flow chart of an example process of processing data at the client device for display in a GUI is illustrated. The method describes at a high level the process followed at the processor of the client device for handling patient data for display in the GUI of the user device. At step 802, the processor displays a subset of stored patient data presented in the GUI of the client device. At step 804, the processor receives a user request to filter the subset of stored patient data. At step 806, the processor updates the subset of stored patient data based on the received user request. In some implementations, the processor generates for display the updated subset of stored patient data in a first and a second presentation mode. At step 808, the processor displays the updated subset of stored patient data. For example, the processor can display the updated subset of patient data in the first presentation mode based on the generation of the data for display. At step 810, the processor receives a user request to display the updated subset of stored patient data in the second presentation mode. The processor then returns to step 802 to display the updated subset of stored patient data in the second presentation mode based on the generation.

As described above, the patient data records can be stored on the processor of a client device for efficient and secure processing of the patient data. The processor can continuously generate the data for display in multiple panel views based on the user selection of filters and subsets of data to provide selected panel views in the GUI without delay for processing. The ability to quickly move between views and filters of the data can make the user interaction with the GUI less cumbersome and less frustrating in comparison to a user having to individually create a graph or plot for each aspect that they would like to investigate. The options in the GUI allow the user to revise the panel view of the patient data quickly.

The processor can enable the quick toggling between available panel views of the large amount of patient data by continuously updating the data for display and associating the data with applied filters as the user selects them. The processor can determine which patient data records are relevant to a particular user selection by comparing text strings within a data array (or “data object”) for each patient data record to a text string associated with a filter selected by a user. When the filter selected by the user is a numeric filter (for example, an age of onset), the processor can determine whether numbers in the data array for each record match the filtered criteria or are within a filtered criteria range.

For example, in some implementations, filtering patient data based on user inputs to the client device includes an analysis of data arrays including one or more text strings to determine whether each patient record includes the filtered characteristic. As described above, the patient data records include data records for a number of patients having a first diagnosis, and each record of the patient data records includes an array of text strings describing aspects of a medical history of a patient. The processor receives a first input to filter the patient data records based on a first criteria including a first text string, and compares the first text string to at least one text string of the array of text strings for each record of the patient data records. Based on the comparison, the processor determines a subset of the patient data records including the first text string, and associates the subset of patient data records including the first text string with a first indicator. The processor then generates for display the subset of the patient data records including the first text string. When the processor receives a second input to filter the subset of patient data records based on a second criteria including a second text string, the processor compares the second text string to at least one text string of the array of text strings for each record of the subset of patient data records associated with the first text string to determine a second subset of patient data records including the second text string among the subset of patient data records associated with the first text string. The processor associates the second subset of patient data records including the second text string with a second indicator, and generates for display the second subset of patient data records including the second string.

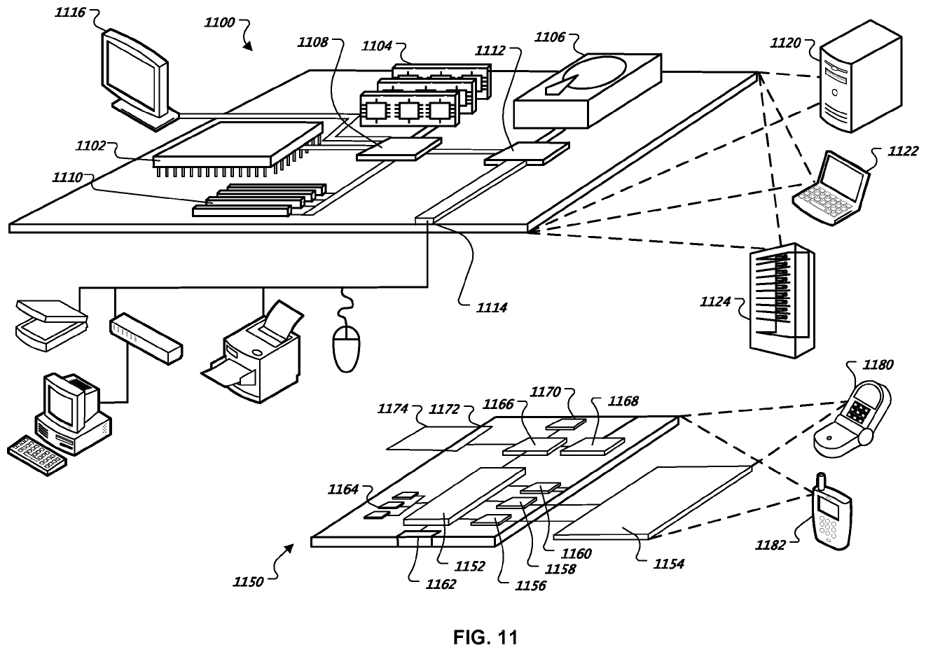

FIG. 11 is a block diagram of computing devices 1100, 1150 that may be used to implement the systems and methods described in this document, either as a client or as a server or plurality of servers. Computing device 1100 is intended to represent various forms of digital computers, such as laptops, desktops, workstations, personal digital assistants, servers, blade servers, mainframes, and other appropriate computers. Computing device 1150 is intended to represent various forms of mobile devices, such as personal digital assistants, cellular telephones, smartphones, and other similar computing devices. Additionally computing device 1100 or 1150 can include Universal Serial Bus (USB) flash drives. The USB flash drives may store operating systems and other applications. The USB flash drives can include input/output components, such as a wireless transmitter or USB connector that may be inserted into a USB port of another computing device. The components shown here, their connections and relationships, and their functions, are meant to be exemplary only, and are not meant to limit implementations described and/or claimed in this document.

Computing device 1100 includes a processor 1102, memory 1104, a storage device 1106, a high-speed interface 1108 connecting to memory 1104 and high-speed expansion ports 1110, and a low speed interface 1112 connecting to low speed bus 1114 and storage device 1106. Each of the components 1102, 1104, 1106, 1108, 1110, and 1112, are interconnected using various busses, and may be mounted on a common motherboard or in other manners as appropriate. The processor 1102 can process instructions for execution within the computing device 1100, including instructions stored in the memory 1104 or on the storage device 1106 to display graphical information for a GUI on an external input/output device, such as display 1116 coupled to high speed interface 1108. In other implementations, multiple processors and/or multiple buses may be used, as appropriate, along with multiple memories and types of memory. Also, multiple computing devices 1100 may be connected, with each device providing portions of the necessary operations (e.g., as a server bank, a group of blade servers, or a multi-processor system).

The memory 1104 stores information within the computing device 1100. In one implementation, the memory 1104 is a volatile memory unit or units. In another implementation, the memory 1104 is a non-volatile memory unit or units. The memory 1104 may also be another form of computer-readable medium, such as a magnetic or optical disk.

The storage device 1106 is capable of providing mass storage for the computing device 1100. In one implementation, the storage device 1106 may be or contain a computer-readable medium, such as a floppy disk device, a hard disk device, an optical disk device, or a tape device, a flash memory or other similar solid-state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 1104, the storage device 1106, or memory on processor 1102.

The high-speed controller 1108 manages bandwidth-intensive operations for the computing device 1100, while the low speed controller 1112 manages lower bandwidth-intensive operations. Such allocation of functions is exemplary only. In one implementation, the high-speed controller 1108 is coupled to memory 1104, display 1116 (e.g., through a graphics processor or accelerator), and to high-speed expansion ports 1110, which may accept various expansion cards (not shown). In the implementation, low-speed controller 1112 is coupled to storage device 1106 and low-speed expansion port 1114. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1100 may be implemented in a number of different forms, as shown in the figure. For example, it may be implemented as a standard server 1120, or multiple times in a group of such servers. It may also be implemented as part of a rack server system 1124. In addition, it may be implemented in a personal computer such as a laptop computer 1122. Alternatively, components from computing device 1100 may be combined with other components in a mobile device (not shown), such as device 1150. Each of such devices may contain one or more of computing device 1100, 1150, and an entire system may be made up of multiple computing devices 1100, 1150 communicating with each other.

Computing device 1150 includes a processor 1152, memory 1164, an input/output device such as a display 1154, a communication interface 1166, and a transceiver 1168, among other components. The device 1150 may also be provided with a storage device, such as a microdrive or other device, to provide additional storage. Each of the components 1150, 1152, 1164, 1154, 1166, and 1168, are interconnected using various buses, and several of the components may be mounted on a common motherboard or in other manners as appropriate.

The processor 1152 can execute instructions within the computing device 1150, including instructions stored in the memory 1164. The processor may be implemented as a chipset of chips that include separate and multiple analog and digital processors. Additionally, the processor may be implemented using any of a number of architectures. For example, the processor 1110 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 1150, such as control of user interfaces, applications run by device 1150, and wireless communication by device 1150.

Processor 1152 may communicate with a user through control interface 1158 and display interface 1156 coupled to a display 1154. The display 1154 may be, for example, a TFT (Thin-Film-Transistor Liquid Crystal Display) display or an OLED (Organic Light Emitting Diode) display, or other appropriate display technology. The display interface 1156 may comprise appropriate circuitry for driving the display 1154 to present graphical and other information to a user. The control interface 1158 may receive commands from a user and convert them for submission to the processor 1152. In addition, an external interface 1162 may be provide in communication with processor 1152, so as to enable near area communication of device 1150 with other devices. External interface 1162 may provide, for example, for wired communication in some implementations, or for wireless communication in other implementations, and multiple interfaces may also be used.

The memory 1164 stores information within the computing device 1150. The memory 1164 can be implemented as one or more of a computer-readable medium or media, a volatile memory unit or units, or a non-volatile memory unit or units. Expansion memory 1174 may also be provided and connected to device 1150 through expansion interface 1172, which may include, for example, a SIMM (Single In Line Memory Module) card interface. Such expansion memory 1174 may provide extra storage space for device 1150, or may also store applications or other information for device 1150. Specifically, expansion memory 1174 may include instructions to carry out or supplement the processes described above, and may include secure information also. Thus, for example, expansion memory 1174 may be provide as a security module for device 1150, and may be programmed with instructions that permit secure use of device 1150. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1164, expansion memory 1174, or memory on processor 1152 that may be received, for example, over transceiver 1168 or external interface 1162.

Device 1150 may communicate wirelessly through communication interface 1166, which may include digital signal processing circuitry where necessary. Communication interface 1166 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1168. In addition, short-range communication may occur, such as using a Bluetooth, WiFi, or other such transceiver (not shown). In addition, GPS (Global Positioning System) receiver module 1170 may provide additional navigation- and location-related wireless data to device 1150, which may be used as appropriate by applications running on device 1150.

Device 1150 may also communicate audibly using audio codec 1160, which may receive spoken information from a user and convert it to usable digital information. Audio codec 1160 may likewise generate audible sound for a user, such as through a speaker, e.g., in a handset of device 1150. Such sound may include sound from voice telephone calls, may include recorded sound (e.g., voice messages, music files, etc.) and may also include sound generated by applications operating on device 1150.

The computing device 1150 may be implemented in a number of different forms, as shown in the figure. For example, it may be implemented as a cellular telephone 1180. It may also be implemented as part of a smartphone 1182, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. Moreover, other mechanisms for performing the systems and methods described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

1. A method for presenting a graphical user interface, the method comprising:
accessing raw patient data on a user device, the user device including a display and an input mechanism;
pre-processing the raw patient data to generate visualization data of a set of patient data records for each of a plurality of panel views;
receiving, at the input mechanism, a first user input selecting a first panel view of the plurality of panel views;
displaying, in a GUI presented on the display of the user device, the set of patient data records in the first panel view;
receiving, at the input mechanism, a second user input selecting a subset of the patient data records;
filtering the pre-processed set of patient data records, on the user device and based on the second user input, to obtain a filtered subset of patient data records; and
displaying, in the GUI, at least the filtered subset of patient data records based on the visualization data.

2. The method of claim 1, wherein the raw patient data, visualization data, and set of patient data records, are stored on the user device.

3. The method of claim 2, further comprising:
updating metadata associated with each record of the filtered subset of patient data records based on the filtering.

4. The method of claim 3, further comprising:
receiving, at the input mechanism, a third user input that selects a second panel view of the plurality of panel views;
generating for display, based on the visualization data, the filtered subset of patient data records in the second panel view; and
displaying, in the GUI, the filtered subset of patient data records in the second panel view.

5. The method of claim 1, wherein the first user input is a selection of a plurality of graphical indicators representing data records in the set of patient records displayed in the first panel view, and displaying at least the filtered subset of patient data records comprises displaying only data records represented by the selected plurality of graphical indicators.

6. (canceled)

7. The method of claim 5, further comprising:
displaying, in the GUI and based on a third user selection received at the input mechanism, additional information related to a selected data record of the filtered subset of patient data records.

8. The method of claim 1, wherein the second user input selects a filtering characteristic, and displaying at least the filtered subset of patient data records comprises displaying the set of patient data records with the filtered subset of patient data records indicated by a visual identifier.

9. (canceled)

10. The method of claim 1, wherein the first panel view comprises one of a cluster view, a time series view, an onset time view, a Sankey plot view, and a SAX visualization view.

11. The method of claim 1, further comprising:
determining a relationship or fit to displayed patient data records in at least one of the plurality of panel views; and
displaying the determined relationship or fit with the displayed patient data records.

12. The method of claim 1, wherein the set of patient data records comprises one or more data records of patients having a first diagnosis, and filtering the set of patient data records further comprises determining a subset of data records of the one or more data records of patients having a first diagnosis that have a first common characteristic.

13. (canceled)

14. The method of claim 12, wherein the first common characteristic is selected from the following list: a genetic marker, a type of diagnosis, age, race, disease state, presence of antibody, or having a second diagnosis, different from the first diagnosis.

15. (canceled)

16. The method of claim 4, wherein the set of patient data records comprises data records of patients with myositis, and the first panel view comprises a cluster plot view.

17. The method of claim 16, wherein the second panel view comprises a time of onset view.

18. The method of claim 17, wherein the filtered subset of patient data records comprises data records of patients with myositis having at least one of diagnosis of cancer, a genetic marker, or an antibody.

19. (canceled)

20. (canceled)

21. The method of claim 4, wherein the set of patient data records comprises data records of patients with chronic obstructive pulmonary disease (COPD), and the first panel view comprises a Sankey plot.

22. The method of claim 21, wherein the second panel view comprises a cluster plot view or a SAX visualization view.

23. The method of claim 22, wherein the filtered subset of patient data records comprises data records of patients with COPD with a history of using a first medication.

24. (canceled)

25. A method of analyzing patient data, the method comprising:
accessing a set of patient data records stored on a user device, the set of patient data records comprising data records for a plurality of patients having a first diagnosis, each data record including a time offset factor to align each of the plurality of patients with a common time zero, and the user device including a display and an input mechanism;
receiving, at the input mechanism, a first input of a first filtering criteria;
determining a subset of the set of patient data records that include the first filtering criteria;
associating each record of the subset of the set of patient data records with the first filtering criteria;
displaying, in a GUI presented in the display of the user device, the set of patient data records in a first panel view based on the common time zero, with the subset of the set of patient data records indicated by a visual indicator;
receiving, at the input mechanism, a second input to display the set of patient data records in a second panel view; and
displaying, in the GUI, the set of patient data records in the second panel view, with the subset of the set of patient data records indicated by the visual indicator based on the association of each record with the first filtering criteria.

26. The method of claim 25, further comprising:
receiving, at the input mechanism, a third input of a second filtering criterion;
determining a second subset of the subset of the set of patient data records that include the second filtering criterion; and
associating the second subset with the second filtering criterion.

27. The method of claim 25, further comprising:
generating, after associating each record with the first filtering criteria, for display the set of patient data records by generating data for display in each of first and second panel views;
displaying, upon receiving a user selection of the first panel view or the second panel view, the data for display in the selected first or second panel view; and
updating the generated data for display based on receiving a further input.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)
